(19) United States
(12) Patent Application Publication
(10) Pub. No.: US 2017/0258437 A1
(43) Pub. Date: Sep. 14, 2017

(54) ULTRASONIC DIAGNOSTIC DEVICE

(71) Applicant: Hitachi, Ltd., Tokyo (JP)

(72) Inventors: Toshinori MAEDA, Tokyo (JP); Eiji KASAHARA, Tokyo (JP); Masaru MURASHITA, Tokyo (JP); Yuko NAGASE, Tokyo (JP); Yuki TANAKA, Tokyo (JP)

(73) Assignee: Hitachi, Ltd., Tokyo (JP)

(21) Appl. No.: 15/509,423

(22) PCT Filed: Aug. 20, 2015
(86) PCT No.: PCT/JP2015/073301

§ 371 Date: Mar. 7, 2017

(30) Foreign Application Priority Data
Sep. 8, 2014 (JP) 2014-181907

(51) Int. Cl. A61B 8/02; A61B 8/08; A61B 8/00

(52) U.S. Cl. CPC A61B 8/02; A61B 8/461; A61B 8/4483; A61B 8/5223; A61B 8/0866

(57) ABSTRACT

A region of interest setting unit sets a region of interest within image data for a tomographic image. The region of interest setting unit sets the region of interest to the heart of an embryo and partitions the region of interest into a plurality of blocks. A waveform generating unit generates an embryonic heartbeat waveform for each block of the plurality of blocks within the region of interest on the basis of the image data within the block. A waveform evaluating unit uses a standard waveform to evaluate the reliability of the embryonic heartbeat waveform for each block of the plurality of blocks within the region of interest.

### TECHNICAL FIELD

The present invention relates to an ultrasonic diagnostic device that diagnoses a fetus (embryo).

### BACKGROUND

Ultrasonic diagnostic devices are used for diagnosis of a tissue within a human body, for example, and are very useful especially in diagnosis of an embryo or a fetus. However, because a fetus (embryo) in the earlier stage of pregnancy, before about 10 weeks, for example, is small and the heart thereof is also very small, diagnosis of the fetal heart using ultrasonic diagnostic devices is extremely difficult. In M-mode measurement and Doppler measurement using an ultrasonic diagnostic device, for example, precise setting of a cursor with respect to the heart having a very small size is difficult. Under these circumstances, various techniques concerning diagnosis of a fetus using an ultrasonic diagnostic device have been proposed. Patent Document 1, for example, proposes an epoch-making technique for obtaining information concerning the fetal heartbeat, from which body shift information has been subtracted, based on motion information of the heart.

### CITATION LIST

### Patent Literature

Patent Document 1: JP 2013-198636 A

### SUMMARY

### Technical Problem

The present invention was made in consideration of the background art described above and is directed to providing an improved technique of ultrasonic diagnostic devices for diagnosing fetal heartbeat.

### Solution to Problem

In accordance with a preferable aspect, an ultrasonic diagnostic device of the present invention includes a probe configured to transmit and receive an ultrasonic wave with respect to a diagnostic region including a fetus, a waveform generating unit configured to generate a heartbeat waveform of the fetus based on data obtained from the diagnostic region via an ultrasonic wave, and a waveform evaluating unit configured to compare the heartbeat waveform of the fetus with a reference waveform having a periodicity to evaluate reliability of the heartbeat waveform of the fetus.

In the above device, the waveform generating unit generates a heartbeat waveform of a fetus (fetal heartbeat waveform) based on image data of an ultrasound image obtained from a diagnosis region including a fetus (embryo) such as a region including the heart of the fetus (fetal heart). For example, the heartbeat waveform may be obtained based on a change of the average value concerning the luminance of an ultrasound image within the region with respect to time, or based on a correlation value between time phases concerning the ultrasound image within the region. Further, the waveform evaluating unit uses, as a reference waveform having a periodicity, a waveform having an amplitude periodically varying in a repeated manner between the positive direction and the negative direction. While a sine wave (cosine wave), for example, is preferable as the reference waveform, a triangular wave, a saw-tooth wave, or a rectangular wave may alternatively be used.

As the above device evaluates the reliability of the fetal heartbeat waveform, a heartbeat waveform with relatively high reliability can be selectively used.

In a specific preferable example, the waveform evaluating unit is configured to evaluate the reliability of the heartbeat waveform based on a correlation between a reference waveform having a period conforming to a period of the heartbeat waveform of the fetus, and the heartbeat waveform.

In a specific preferable example, the waveform evaluating unit is configured to calculate an evaluation value related to the reliability of the heartbeat waveform based on a cross-correlation function of the heartbeat waveform of the fetus and the reference waveform.

In a specific preferable example, a region including the heart of the fetus is divided into a plurality of blocks. The waveform generating unit is configured to generate the heartbeat waveform of the fetus for each block of the plurality of blocks, based on data obtained from each block. The waveform evaluating unit is configured to evaluate the reliability of the heartbeat waveform for each block of the plurality of blocks.

In a specific preferable example, the waveform evaluating unit is configured to calculate an evaluation value related to the reliability of the heartbeat waveform for each block and to select a representative heartbeat waveform from among a plurality of heartbeat waveforms corresponding to the plurality of blocks, based on the evaluation value calculated for each block.

In a specific preferable example, the waveform generating unit is configured to calculate, for each block of the plurality of blocks, an average luminance within each block based on data obtained from each block, and to generate the heartbeat waveform having an amplitude corresponding to the average luminance.

In a specific preferable example, the waveform evaluating unit is configured to use an appropriate peak other than an inappropriate peak, among a plurality of peaks detected within the heartbeat waveform of the fetus, to calculate a period of the heartbeat waveform, and to use the reference waveform having a period identical to the period that is calculated.

In a specific preferable example, the waveform evaluating unit is configured to designate each of the plurality of peaks that are detected as a noted point. If another peak exits within a determination time range corresponding to a noted point, and the other peak has an average luminance higher than an average luminance of the noted point, the waveform evaluating unit determines the noted point as the inappropriate peak.

In a specific preferable example, the waveform evaluating unit is configured to sequentially obtain the cross-correlation function while moving the reference waveform with respect to the heartbeat waveform stepwise in a time axis direction or moving the heartbeat waveform with respect to the reference waveform stepwise in the time axis direction, thereby calculating a root mean square of the cross-correlation function, as the evaluation value.

### ADVANTAGEOUS EFFECTS OF INVENTION

Embodiments of the present invention provide an improved technique of an ultrasonic diagnostic device for diagnosing fetal heartbeats. According to a preferable embodiment, for example, as the reliability of a fetal heartbeat waveform is evaluated, a heartbeat waveform with relatively high reliability can be selectively used.

### BRIEF DESCRIPTION OF DRAWINGS

FIG. 1 is a diagram illustrating the overall structure of an ultrasonic diagnostic device preferable in implementation of the invention.

FIG. 2 is a diagram illustrating an example for setting a region of interest.

FIG. 3 is a diagram illustrating an example region of interest which is divided.

FIG. 4 is a diagram illustrating a specific heartbeat waveform.

FIG. 5 is a diagram for explaining example derivation of a period of a heartbeat waveform.

FIG. 6 is a diagram for explaining evaluation of a heartbeat waveform using a reference waveform.

FIG. 7 is a diagram for explaining a cross-correlation function and a calculation example of an evaluation value.

### DESCRIPTION OF EMBODIMENTS

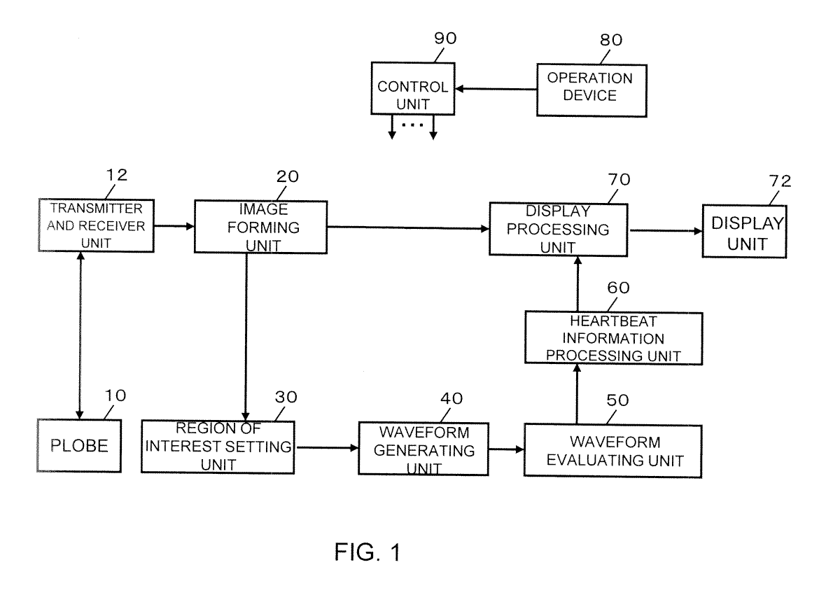

FIG. 1 is a diagram illustrating a whole structure of an ultrasonic diagnostic device according to a preferred embodiment of the present invention. A probe 10 is an ultrasound probe which transmits and receives ultrasonic waves to and from a diagnostic region including a fetus. The probe 10 includes a plurality of transducer elements for transmitting and receiving ultrasonic waves. The plurality of transducer elements are transmission-controlled by a transmitter and receiver unit 12 to form a transmitting beam. The plurality of transducer elements further receive ultrasonic waves from the diagnostic region, and output a signal obtained from the received ultrasonic waves to the transmitter and receiver unit 12. The transmitter and receiver unit 12 then forms a received beam to obtain a received signal (echo data). To transmit and receive the ultrasonic waves, a technique, such as transmission aperture synthesis, may be used.

An image forming unit 20, based on the received signal obtained from the transmitter and receiver unit 12, forms image data of an ultrasound image. The image forming unit 20 applies, to the received signal, signal processing including gain correction, logarithmic compression, wave detection, contour enhancement, filter processing, and other processing, as necessary, to form, for example, image data of a tomographic image (B-mode image) showing a fetus, for each of a plurality of frames (for each time phase).

The image data of a tomographic image formed in the image forming unit 20 are output to a region of interest setting unit 30. The image data formed in the image forming unit 20 also undergo display processing in a display processing unit 70, and a tomographic image corresponding to the image data is displayed on a display unit 72.

The region of interest setting unit 30 sets a region of interest within the image data of a tomographic image formed in the image forming unit 20. The region of interest setting unit 30 sets the region of interest to the fetal heart. The region of interest setting unit 30 further divides the region of interest into a plurality of blocks.

After the region of interest is set, a waveform generating unit 40 forms a fetal heartbeat waveform, based on the image data within the region of interest. The waveform generating unit 40 generates the fetal heartbeat waveform for each of the plurality of blocks in the region of interest, based on the image data in the block.

When the heartbeat waveform is generated, a waveform evaluating unit 50 evaluates the reliability of the heartbeat waveform. The waveform evaluating unit 50 evaluates the reliability of the heartbeat waveform (e.g., stability of the waveform) for each of the plurality of blocks within the region of interest.

Processing performed by the region of interest setting unit 30, the waveform generating unit 40, and the waveform evaluating unit 50 will be described in detail below.

A heartbeat information processing unit 60 obtains fetal heartbeat information based on, for example, a heartbeat waveform having relatively high reliability. The heartbeat information obtained in the heartbeat information processing unit 60 is displayed, via the display processing unit 70, on the display unit 72.

A control unit 90 controls the whole ultrasonic diagnostic device illustrated in FIG. 1. An instruction received by a user via an operation device 80 is also reflected in the whole control performed by the control unit 90.

Among the elements (units designated by reference numerals) illustrated in FIG. 1, each of the transmitter and receiver unit 12, the image forming processing unit 20, the region of interest setting unit 30, the waveform generating unit 40, the waveform evaluating unit 50, the heartbeat information processing unit 60, and the display processing unit 70 may be implemented by using hardware such as an electrical or electronic circuit or a processor, for example, and a device such as a memory may be used as required for the implementation. Further, functions corresponding to the respective units described above may be implemented by cooperation of hardware such as a CPU, a processor, and a memory, and software (a program) which regulates the operation of the CPU or the processor.

A preferable specific example of the display unit 72 is a liquid crystal display, for example. The operation device 80 can be implemented by at least one of a mouse, a keyboard, a trackball, a touch panel, and other switches. The control unit 90 can be implemented by cooperation of hardware such as a CPU, a processor, and a memory, and software (a program) which regulates the operation of the CPU or the processor.

The whole structure of the ultrasonic diagnostic device illustrated in FIG. 1 has been described as above. Specific example processing in the ultrasonic diagnostic device will now be described in detail. The following description concerning the elements (units denoted by reference numerals) illustrated in FIG. 1 uses the reference numerals in FIG. 1.

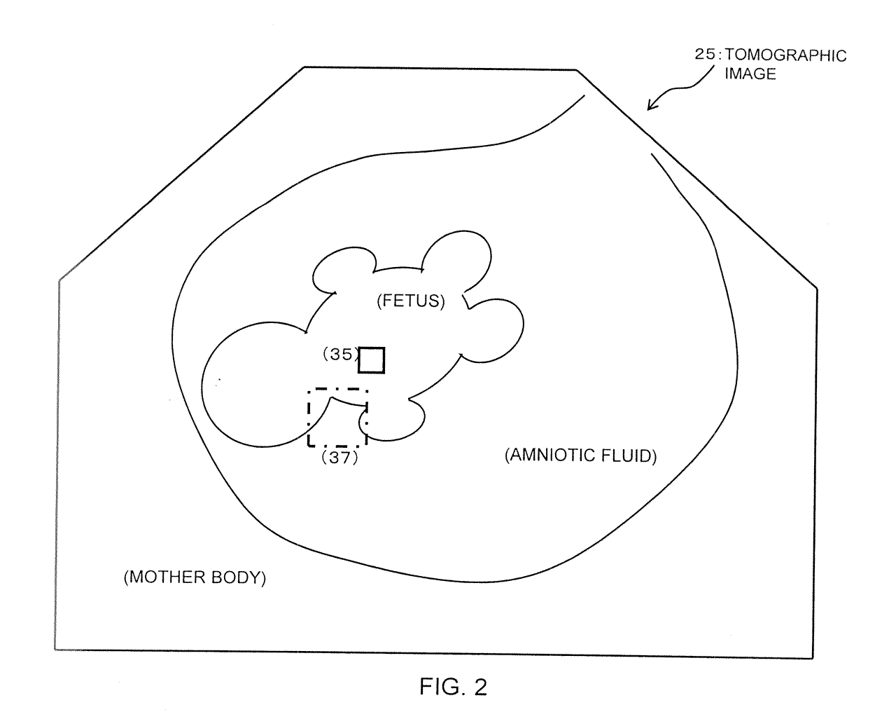

FIG. 2 is a diagram illustrating an example for setting a region of interest 35. The region of interest setting unit 30 sets the region of interest 35 within a tomographic image (image data) 25 formed by the image forming unit 20. The tomographic image 25 shows a fetus within a mother's body (the uterus), and the fetus is surrounded by amniotic fluid within the mother's body.

The region of interest setting unit 30 sets the region of interest 35 with respect to the fetal heart. The region of interest setting unit 30, for example, sets the region of interest 35 in accordance with a user operation input via the operation device 80. The user operates the operation device 80 to set the region of interest 35 such that the region of interest 35 includes the fetal heart (particularly, the heart wall), for example, while observing the tomographic image 25 displayed on the display unit 72. The region of interest setting unit 30 may analyze the image state within the tomographic image 25 to set the region of interest 35 to the fetal heart.

The region of interest 35 is used for diagnosis of the fetal heartbeat and is therefore preferably set to a location where the motion of the fetal heart can be easily detected. More specifically, the user designates the location of the region of interest 35 such that a portion of the fetal heart having a relatively high luminance, and more preferably the heart wall, is included in the region of interest 35. Further, the ultrasonic diagnostic device illustrated in FIG. 1 may determine a portion of the fetal heart having a relatively high luminance based on image analysis processing such as binarization processing, for example, to thereby determine the location of the region of interest 35. The region of interest 35 may be set to other portions where the motion of the fetal heart can be easily detected.

While in the specific example illustrated in FIG. 2, the region of interest 35 has a rectangular shape, the region of interest 35 may be of other polygonal shape, or a circle or ellipse. In addition to the region of interest 35, a body reference region 37 may be set as in the specific example of FIG. 2. For example, as described in Patent Document 1 (JP 2013-198636 A), the motion of a fetal body may be analyzed using the body reference region 37 to obtain body change information, which is then to be subtracted from the information concerning the fetal heartbeat obtained based on the region of interest 35. For example, the region of interest 35 in the heart may be moved to follow the motion of the fetal body based on the body change information obtained using the body reference region 37.

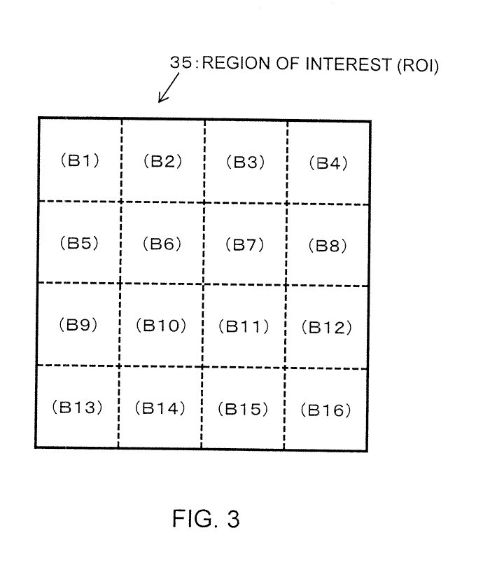

FIG. 3 is a diagram illustrating an example region of interest 35 which is divided. The region of interest setting unit 30 divides the region of interest 35 into a plurality of blocks. FIG. 3 illustrates a region of interest 35 of a rectangular shape, which is divided into 16 blocks (B1 to B16). The example division of the region of interest 35 illustrated in FIG. 3 is only one specific example, and the region of interest 35 may be divided into a plurality of blocks other than 16 blocks, and the shape of each block is not limited to a rectangle. Some of the blocks may overlap each other. When a tomographic image of the enlarged heart is obtained, the whole tomographic image may be considered the region of interest 35 and divided into a plurality of blocks.

After the region of interest is set, the waveform generating unit 40 generates a fetal heartbeat waveform based on the image data within the region of interest. The waveform generating unit 40, for each of the plurality of blocks (B1 to B16) within the region of interest 35 illustrated in FIG. 3, generates a fetal heartbeat waveform based on the image data within the block.

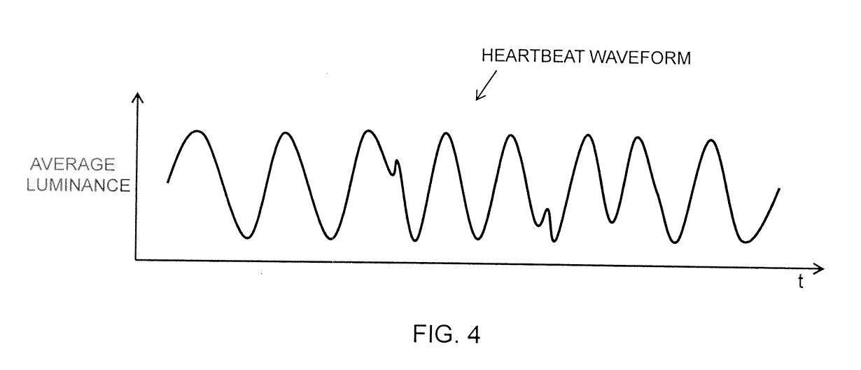

FIG. 4 is a chart illustrating a specific example heartbeat waveform. FIG. 4 illustrates a heartbeat waveform indicating the average luminance, which is an amplitude, on the vertical axis with the horizontal axis being a time axis.

The waveform generating unit 40 calculates the average luminance (average of the luminance values) for each of the blocks within the region of interest, based on the image data within the block, and calculates the average luminance over a plurality of times, thereby generating, for each block, the heartbeat waveform as illustrated in FIG. 4. Due to a periodical expansion and contraction motion of the fetal heart, the average luminance within each block varies with the expansion and contraction motion, and therefore the heartbeat waveform as in the specific example illustrated in FIG. 4, for example, is obtained.

In place of the average luminance, a correlation value between time phases of the image data may be used to generate the heartbeat waveform. For example, the waveform generating unit 40 may calculate, for each block, a correlation value between the image data at the reference time phase and the image data at each time phase over a plurality of time phases to generate a heartbeat waveform, with the correlation values being the amplitude on the vertical axis. The waveform generating unit 40 may form a heartbeat waveform based on Doppler information, for example, for each block.

After a heartbeat waveform is formed, the waveform evaluating unit 50 compares the heartbeat waveform with the reference waveform to evaluate the reliability of the heartbeat waveform. The waveform evaluating unit 50 evaluates the reliability of the heartbeat waveform for each of the plurality of blocks (B1 to B16) within the region of interest 35 illustrated in FIG. 3, for example. To evaluate the heartbeat waveform, the waveform evaluating unit 50 first derives the period of the heartbeat waveform.

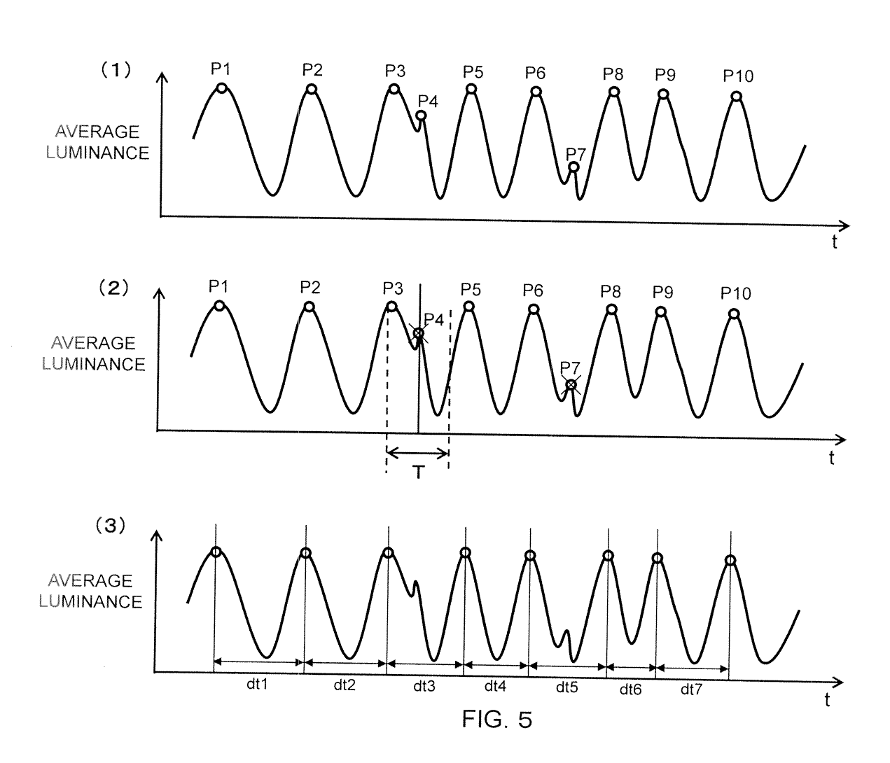

FIG. 5 is a diagram for explaining an example of deriving the period of a heartbeat waveform. First, processing using a low-pass filter, for example, is applied to the original heartbeat waveform (FIG. 4) for removing micro irregularities (noise) within the heartbeat waveform, such that there can be obtained the heartbeat waveform illustrated in FIG. 5(1), with micro irregularities being removed while maintaining the periodical characteristic of the original heartbeat waveform (FIG. 4).

The waveform evaluating unit 50 then locates a peak (maximum point) in the heartbeat waveform in FIG. 5(1). A noted point on the heartbeat waveform having the average luminance which is higher than the average luminance of an adjacent point before or after the noted point (or a vicinity point before or after the noted point), for example, is determined as a peak (maximum point). In this manner, the peaks are detected over the whole region of the heartbeat waveform. FIG. 5(1) illustrates a plurality of peaks (P1 to P10) detected in the heartbeat waveform.

The waveform evaluating unit 50 further locates a peak which is improper in calculation of the period, among the plurality of peaks (P1 to P10) detected in the heartbeat waveform. For example, concerning each of noted points designated by the detected plurality of peaks (P1 to P10), when another peak having an average luminance higher than that of the noted point exists within a determination time range T with the noted point being the center thereof, the noted point is determined as an improper peak. With this processing, a peak P4 and a peak P7, among the plurality of peaks (P1 to P10), are determined as improper peaks as in the specific example illustrated in FIG. 5(2), for example.

The waveform evaluating unit 50 then calculates the period (heart rate) of the heartbeat waveform using proper peaks other than the improper peaks. As illustrated in the specific example in FIG. 5(3), the period of the heartbeat waveform is calculated based on a plurality of peak intervals (dt1 to dt7) obtained only from the proper peaks.

The waveform evaluating unit 50 sets the average value of the plurality of peak intervals (dt1 to dt7) as the period of the heartbeat waveform. The waveform evaluating unit 50 may also set the average value of the plurality of peak intervals (dt1 to dt7) as a temporary average value, and, after removing peak intervals, among the plurality of peak intervals (dt1 to dt7), deviated from the temporary average value by a significant amount (difference from the temporary average value being equal to or greater than a determination threshold value), calculate a true average value from the remaining plurality of peak intervals and set the true average value as the period of the heartbeat waveform. In the specific example illustrated in FIG. 5(3), for example, with the peak intervals dt1 and dt6, which are determined to be significantly deviated from the temporary average value, being removed, a true average value obtained from the remaining plurality of peak intervals may be set as the period of the heartbeat waveform.

The waveform evaluating unit 50 may obtain the period of the heartbeat waveform using the minimum points within the heartbeat waveform along with or in place of the maximum points within the heartbeat waveform.

Obtaining the period of the heartbeat waveform, the waveform evaluating unit 50 then evaluates the heartbeat waveform using the reference waveform.

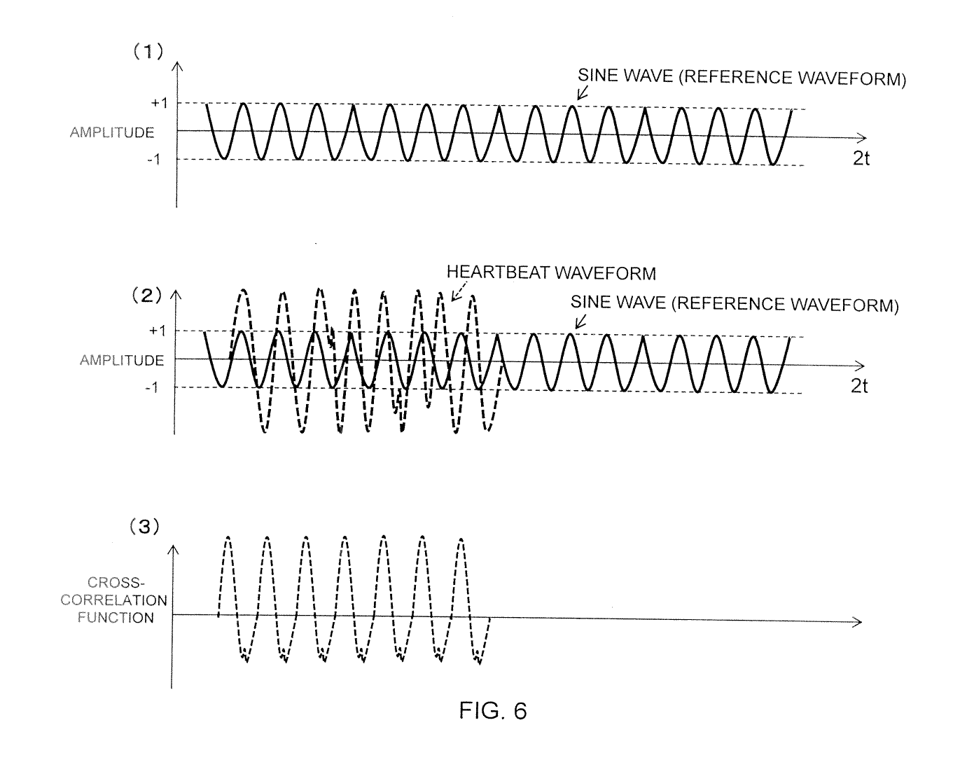

FIG. 6 is a diagram for explaining evaluation of a heartbeat waveform using a reference waveform. The waveform evaluating unit 50 compares a heartbeat waveform with a reference waveform having the same period as that of the heartbeat waveform and calculates an evaluation value concerning the reliability of the heartbeat waveform. The waveform evaluating unit 50 uses, as the reference waveform, the sine wave shown in FIG. 6(1).

The waveform evaluating unit 50 sets the period of the sine wave, which is the reference waveform, to a period which is the same as that of the heartbeat waveform, and compares the reference waveform and the heartbeat waveform with each other. FIG. 6(2) illustrates a heartbeat waveform and a sine wave (reference waveform) having the period conforming to the period of the heartbeat waveform. The heartbeat waveform to be evaluated may be the original heartbeat waveform (FIG. 4) of the heartbeat waveform having been subjected to processing using a low-pass filter, for example (FIG. 5(1)). The amplitude of the sine wave, which is the reference waveform, is set from plus 1 (+1) to minus 1(−1), and the length of the sine wave in the direction of the time axis is set to twice that of the heartbeat waveform.

The waveform evaluating unit 50 then obtains the cross-correlation function illustrated in FIG. 6(3) based on the sine wave which is the reference waveform and the heartbeat waveform, and calculates an evaluation value concerning the reliability of the heartbeat waveform.

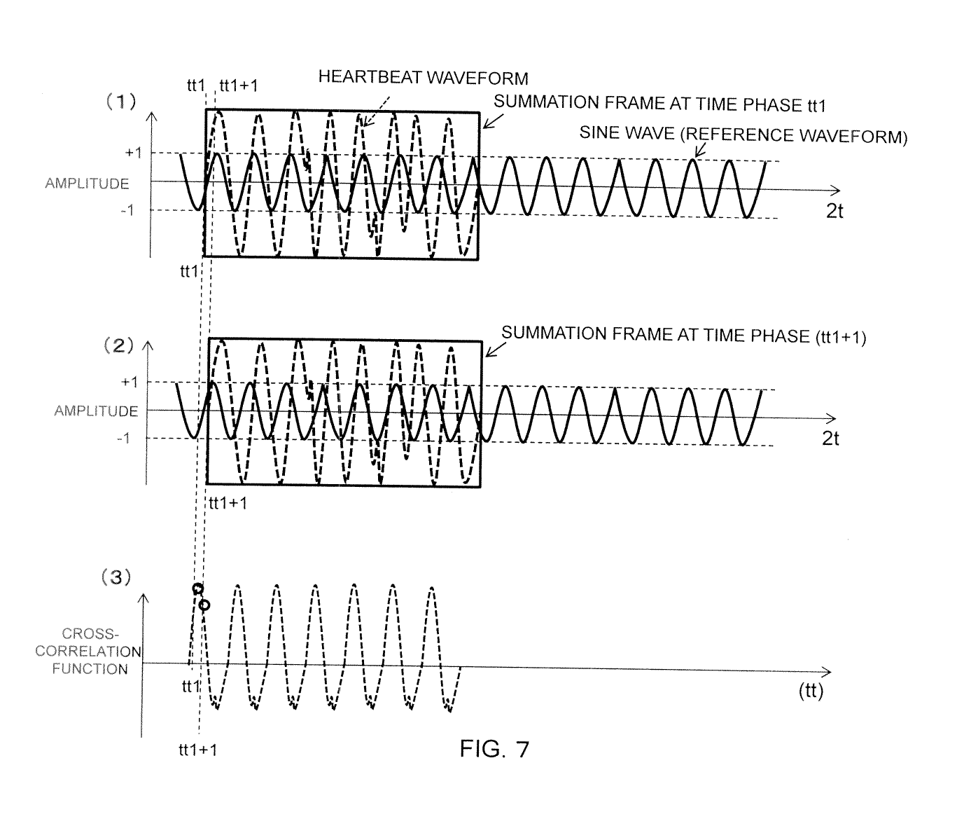

FIG. 7 is a diagram for explaining an example for calculating the cross-correlation function and the evaluation values. The waveform evaluating unit 50 uses the sine wave which is the reference waveform and the heartbeat waveform to calculate the cross-correlation function based on Mathematical Formula 1. In Mathematical Formula 1, f(t) is a heartbeat waveform, and sin (tt+t) is a sine wave (reference waveform).

$$cross \text{-} reference function ( tt ) = \frac{1}{N} {\sum }_{t = 0}^{t = N - 1} f ( t ) \cdot sin ( tt + t ) \qquad [ Mathematical Formula 1 ]$$

FIG. 7 illustrates a calculation example of the cross-correlation function based on Mathematical Formula 1. FIG. 7(1) illustrates a heartbeat waveform and a sine wave (reference waveform), and also a summation frame of the cross-correlation function at a time phase ttl. Specifically, to calculate the cross-correlation function (ttl) at the time phase tt1 (tt=tt1) in Mathematical Formula 1, summation (E) concerning time tin Mathematical Formula 1 is executed in the summation frame in FIG. 7(1), thereby calculating the cross-correlation function (tt1) at the time phase tt1.

FIG. 7(2) illustrates, along with the heartbeat waveform and the sine wave, a summation frame of the cross-correlation function at the time phase tt1+1. To calculate the cross-correlation function (tt1+1) at the time phase tt1+1 (tt=tt1+1) in Mathematical Formula 1, summation (E) concerning time tin Mathematical Formula 1 is executed in the summation frame in FIG. 7(2), thereby calculating the cross-correlation function (tt1+1) at the time phase tt1+1.

The waveform evaluating unit 50 similarly shifts the summation frame stepwise for each time phase after the time phase tt1+2, thereby sequentially calculating the cross-correlation function (tt). Consequently, the cross-correlation function as shown in a specific example in FIG. 7(3) is obtained.

The waveform evaluating unit 50 further calculates a root mean square value (RMS) of the cross-correlation function based on Mathematical Formula 2.

$$RMS = \sqrt{\frac{1}{N} {\sum }_{tt = 0}^{tt = N - 1} cross \text{-} reference function {( tt )}^{2}} \qquad [ Mathematical Formula 2 ]$$

In applying the heartbeat waveform f(t) to Mathematical Formula 1, an offset of the heartbeat waveform f(t) is preferably removed. With the example heartbeat waveform f(t) shown in Mathematical Formula 3, for example, the waveform f″ (t) obtained by second differentiation of the heartbeat waveform f(t) corresponds to a result obtained by multiplying the amplitude of the original waveform f(t) by −a² and removing an offset therefrom. Therefore, the waveform f″ (t) obtained by second differentiation may be multiplied by −1, for example, to align the phase thereof with that of the original waveform f(t), and the resulting waveform may be used as the heartbeat waveform f(t) in Mathematical Formula 1.

$$f ( t ) = A \cdot sin ( at + b ) + offset \\ {f}^{′} ( t ) = a \cdot A \cdot cos ( at + b ) \\ \begin{matrix}{f}^{″} ( t ) = - {a}^{2} \cdot A \cdot sin ( at + b ) \\ - {a}^{2} \cdot f ( t )\end{matrix} \qquad [ Mathematical Formula 3 ]$$

The waveform evaluating unit 50, for each of the plurality of blocks (B1 to B16) within the region of interest 35 shown in FIG. 3, for example, calculates the cross-correlation function of the heartbeat waveform and the sine wave (reference waveform) based on Mathematical Formula 1, and further calculates a root mean square value (RMS) of the cross-correlation function as an evaluation value of each block based on Mathematical Formula 2.

Among the plurality of blocks, a heartbeat waveform having a relatively high reliability is selected as a representative heartbeat waveform based on the evaluation value of the heartbeat waveform calculated for each block. For example, a heartbeat waveform with the maximum RMS obtained by Mathematical Formula 2 is designated as a representative heartbeat waveform.

The heartbeat information processing unit 60, based on the representative heartbeat waveform, for example, calculates the heart rate of a fetus as fetal heartbeat information. The heartbeat information processing unit 60 may select, in addition to or in place of the representative heartbeat waveform, at least one heartbeat waveform with relatively high reliability to calculate the fetal heart rate and other information based on the selected heartbeat waveform. The heartbeat information obtained by the heartbeat information processing unit 60, such as the fetal heart rate, is displayed on the display unit 72 via the display processing unit 70.

The display processing unit 70 further forms a display image of the representative heartbeat waveform for display on the display unit 72. The display processing unit 70 may cause the display unit 72 to display, in addition to or in place of the representative heartbeat waveform, at least one heartbeat waveform from among the plurality of blocks (B1 to B16 in FIG. 3).

While embodiments of the present invention have been described, the embodiments described above are only illustrative in all respects, and do not limit the scope of the invention. The present invention includes various modifications without departing from its spirit.

### REFERENCE SIGNS LIST

10 probe, 12 transmitter and receiver unit, 20 image forming unit, 30 region of interest setting unit, 40 waveform generating unit, 50 waveform evaluating unit, 60 heartbeat information processing unit, 70 display processing unit, 72 display unit, 80 operation device, 90 control unit.

1. An ultrasonic diagnostic device, comprising:
a probe configured to transmit and receive an ultrasonic wave with respect to a diagnostic region including a fetus;
a waveform generating unit configured to generate a heartbeat waveform of the fetus based on data obtained from the diagnostic region via an ultrasonic wave; and
a waveform evaluating unit configured to compare the heartbeat waveform of the fetus with a reference waveform having a periodicity to evaluate reliability of the heartbeat waveform of fetus.

2. The ultrasonic diagnostic device according to claim 1, wherein
the waveform evaluating unit is configured to evaluate the reliability of the heartbeat waveform based on a correlation between a reference waveform having a period conforming to a period of the heartbeat waveform of the fetus, and the heartbeat waveform.

3. The ultrasonic diagnostic device according to claim 1, wherein
the waveform evaluating unit is configured to calculate an evaluation value related to the reliability of the heartbeat waveform based on a cross-correlation function of the heartbeat waveform of the fetus and the reference waveform.

4. The ultrasonic diagnostic device according to claim 2, wherein
the waveform evaluating unit is configured to calculate an evaluation value related to the reliability of the heartbeat waveform based on a cross-correlation function of the heartbeat waveform of the fetus and the reference waveform.

5. The ultrasonic diagnostic device according to claim 1, wherein
a region including the heart of the fetus is divided into a plurality of blocks,
the waveform generating unit is configured to generate the heartbeat waveform of the fetus for each block of the plurality of blocks, based on data obtained from each block, and
the waveform evaluating unit is configured to evaluate the reliability of the heartbeat waveform for each block of the plurality of blocks.

6. The ultrasonic diagnostic device according to claim 5, wherein
the waveform evaluating unit is configured to calculate an evaluation value related to the reliability of the heartbeat waveform for each block and to select a representative heartbeat waveform from among a plurality of heartbeat waveforms corresponding to the plurality of blocks, based on the evaluation value calculated for each block.

7. The ultrasonic diagnostic device according to claim 4,
a region including the heart of the fetus is divided into a plurality of blocks,
the waveform generating unit is configured to generate the heartbeat waveform of the fetus for each block of the plurality of blocks based on data obtained from each block, and
the waveform evaluating unit is configured to evaluate the reliability of the heartbeat waveform for each block of the plurality of blocks.

8. The ultrasonic diagnostic device according to claim 7,
the waveform evaluating unit is configured to calculate the evaluation value related to the reliability of the heartbeat waveform for each block, and to select a representative heartbeat waveform from among a plurality of heartbeat waveforms corresponding to the plurality of blocks based on the evaluation value calculated for each block.

9. The ultrasonic diagnostic device according to claim 5, wherein
the waveform generating unit is configured to calculate, for each block of the plurality of blocks, an average luminance within each block based on data obtained from each block, and to generate the heartbeat waveform having an amplitude corresponding to the average luminance

10. The ultrasonic diagnostic device according to claim 7, wherein
the waveform generating unit is configured to calculate, for each block of the plurality of blocks, an average luminance within each block based on data obtained from each block, and to generate the heartbeat waveform having an amplitude corresponding to the average luminance

11. The ultrasonic diagnostic device according to claim 2,
the waveform evaluating unit is configured to use an appropriate peak other than an inappropriate peak, among a plurality of peaks detected within the heartbeat waveform of the fetus, to calculate a period of the heartbeat waveform, and to use the reference waveform having a period identical to the period that is calculated.

12. The ultrasonic diagnostic device according to claim 11, wherein
the waveform evaluating unit is configured to designate each of the plurality of peaks that are detected as a noted point, and, based on presence of another peak within a determination time range corresponding to a noted point, the other peak having an average luminance higher than an average luminance of the noted point, to determine the noted point as the inappropriate peak.

13. The ultrasonic diagnostic device according to claim 3, wherein
the waveform evaluating unit is configured to sequentially obtain the cross-correlation function while moving the reference waveform with respect to the heartbeat waveform stepwise in a time axis direction or moving the heartbeat waveform with respect to the reference waveform stepwise in the time axis direction, thereby calculating a root mean square of the cross-correlation function, as the evaluation value.

14. The ultrasonic diagnostic device according to claim 4, wherein
the waveform evaluating unit is configured to sequentially obtain the cross-correlation function while moving the reference waveform with respect to the heartbeat waveform stepwise in a time axis direction or moving the heartbeat waveform with respect to the reference waveform stepwise in the time axis direction, thereby calculating a mean square root of the cross-correlation function, as the evaluation value.
